(19) United States
(12) Patent Application Publication
(10) Pub. No.: US 2023/0397883 A1
(43) Pub. Date: Dec. 14, 2023

(54) IMPLANTABLE FILTER REGULATION

(71) Applicant: Cochlear Limited, Macquarie University, NSW (AU)

(72) Inventors: Adam HERSBACH, Richmond, VIC (AU); Thomas LEROUX, Valbonne (FR); Alberto GOZZI, Woluwe-Saint-Pierre (BE)

(21) Appl. No.: 18/251,945

(22) PCT Filed: Oct. 14, 2021
(86) PCT No.: PCT/IB2021/059454

§ 371 Date: May 5, 2023

Related U.S. Application Data
(60) Provisional application No. 63/113,415, filed on Nov. 13, 2020.

(51) Int. Cl. A61B 5/00; A61N 1/36; H04R 25/00; H04R 29/00

(52) U.S. Cl. CPC A61B 5/6814; A61N 1/36038; H04R 25/407; H04R 29/006; A61B 2562/0219; H04R 2460/13

(57) ABSTRACT

An implantable medical device is configured to detect signals with first and second implantable sensors configured to be implanted in a recipient. The implantable medical device is configured to adaptively equalize a response of the first implantable sensor to a response of a similar external sensor, wherein adaption control of the equalization is based on a coherence between the signals detected by first implantable sensor and the signals detected by the external microphone indicating the presence of acoustic signals. In addition, the implantable medical device is configured to adaptively filter vibration signals, including body noises, from the implantable sound signals, wherein adaption control of the filter is based on a coherence between the signals detected by first implantable sensor and the signals detected by the second implantable sensor indicating the presence of vibration.

### BACKGROUND

### Field of the Invention

The present invention relates generally to the regulation of filters associated with implantable sensor.

### Related Art

Medical devices have provided a wide range of therapeutic benefits to recipients over recent decades. Medical devices can include internal or implantable components/devices, external or wearable components/devices, or combinations thereof (e.g., a device having an external component communicating with an implantable component). Medical devices, such as traditional hearing aids, partially or fully-implantable hearing prostheses (e.g., bone conduction devices, mechanical stimulators, cochlear implants, etc.), pacemakers, defibrillators, functional electrical stimulation devices, and other medical devices, have been successful in performing lifesaving and/or lifestyle enhancement functions and/or recipient monitoring for a number of years.

The types of medical devices and the ranges of functions performed thereby have increased over the years. For example, many medical devices, sometimes referred to as “implantable medical devices,” now often include one or more instruments, apparatus, sensors, processors, controllers or other functional mechanical or electrical components that are permanently or temporarily implanted in a recipient. These functional devices are typically used to diagnose, prevent, monitor, treat, or manage a disease/injury or symptom thereof, or to investigate, replace or modify the anatomy or a physiological process. Many of these functional devices utilize power and/or data received from external devices that are part of, or operate in conjunction with, implantable components.

### SUMMARY

In one aspect, a method is provided. The method comprises: detecting signals with an implantable microphone configured to be implanted in a recipient; detecting signals with an implantable vibration sensor configured to be implanted in the recipient; adaptively equalizing a response of the implantable microphone to a response of an external microphone, where the adaptation is controlled by a coherence between the signals detected by the implantable microphone and signals detected by the external microphone; and adaptively filtering vibration signals from the signals detected by the implantable microphone, where the adaptation is controlled by a coherence between the signals detected by the implantable microphone and the signals detected by the implantable vibration sensor.

In another aspect, an apparatus is provided. The apparatus comprises: a first implantable sensor configured to capture signals comprising acoustic sounds and vibration signals; a second implantable sensor configured to capture signals comprising at least vibration signals; an implantable sound processing module configured to filter the vibration signals from the implantable sound signals based on a coherence between the signals captured by the first implantable sensor and the signals captured by the second implantable sensor to generate output signals; and an implantable stimulator unit configured to generate, based on the outputs signals, stimulation signals for delivery to a recipient of the apparatus to evoke perception by the recipient of the acoustic sounds.

In another aspect, one or more non-transitory computer readable storage media are provided. The non-transitory computer readable storage comprises instructions that, when executed by at least one processor, are operable to: obtain signals detected by an implantable microphone configured to be implanted in a recipient; obtain signals detected by an implantable vibration sensor configured to be implanted in the recipient; and adaptively equalize a response of the implantable microphone to a response of an external microphone based on a coherence between the signals detected by the implantable microphone and the signals detected by the external microphone.

In another aspect, one or more non-transitory computer readable storage media are provided. The non-transitory computer readable storage comprises instructions that, when executed by at least one processor, are operable to: obtain signals detected by an implantable microphone configured to be implanted in a recipient, wherein the signals detected by the implantable microphone comprise acoustic sounds and vibration signals; obtain signals detected by an implantable vibration sensor configured to be implanted in the recipient; and filter the vibration signals from the signals detected by the implantable microphone based on a coherence between the signals detected by the implantable microphone and the signals detected by the implantable vibration sensor.

### BRIEF DESCRIPTION OF THE DRAWINGS

Embodiments of the present invention are described herein in conjunction with the accompanying drawings, in which:

FIG. 1A illustrates a cochlear implant system, in accordance with certain embodiments presented herein;

FIG. 1B is a side view of a recipient wearing a sound processing unit of the cochlear implant system of FIG. 1A;

FIG. 1C is a schematic view of components of the cochlear implant system of FIG. 1A;

FIG. 1D is a block diagram of the cochlear implant system of FIG. 1A;

FIG. 2 is a functional block diagram of an implantable sound processing module, in accordance with certain embodiments presented herein;

FIGS. 3A, 3B, and 3C are graphs illustrating a relatively high coherence between an implantable sound signal and an implantable vibration signal, in accordance with certain embodiments presented herein;

FIGS. 4A, 4B, and 4C are graphs illustrating a relatively low coherence between an implantable sound signal and an implantable vibration signal, in accordance with certain embodiments presented herein;

FIG. 5A is a schematic block diagram illustrating operation of an implantable sound processing module of a cochlear implant, in accordance with embodiments presented herein;

FIG. 5B is a schematic block diagram illustrating further details of the noise cancellation of FIG. 5A.

FIG. 6 is a flowchart of an example method, in accordance with certain embodiments presented herein.

### DETAILED DESCRIPTION

Presented herein are techniques for regulation/adjustment of filters associated with implantable sensors of an implantable medical device. In particular, the implantable medical device is configured to detect/capture signals with a first implantable sensor configured to be implanted in a recipient and to capture signals with a second implantable sensor configured to be implanted in the recipient. The implantable medical device is configured to adaptively equalize a response of the first implantable sensor to a response of a similar external sensor, wherein adaption control of the equalization is based on a coherence between the signals detected by first implantable sensor and the signals detected by the external microphone indicating the presence of acoustic signals. In addition, the implantable medical device is configured to adaptively filter vibration signals, including body noises, from the implantable sound signals, wherein adaption control of the filter is based on a coherence between the signals detected by first implantable sensor and the signals detected by the second implantable sensor indicating the presence of vibration.

Merely for ease of description, the techniques presented herein are primarily described with reference to a specific implantable medical device system, namely a cochlear implant system. However, it is to be appreciated that the techniques presented herein may also be implemented by other types of implantable medical devices. For example, the techniques presented herein may be implemented by other auditory prosthesis systems that include one or more other types of auditory prostheses, such as middle ear auditory prostheses, bone conduction devices, direct acoustic stimulators, electro-acoustic prostheses, auditory brain stimulators, etc. The techniques presented herein may also be used with tinnitus therapy devices, vestibular devices (e.g., vestibular implants), visual devices (i.e., bionic eyes), sensors, pacemakers, drug delivery systems, defibrillators, functional electrical stimulation devices, catheters, seizure devices (e.g., devices for monitoring and/or treating epileptic events), sleep apnea devices, electroporation devices, etc.

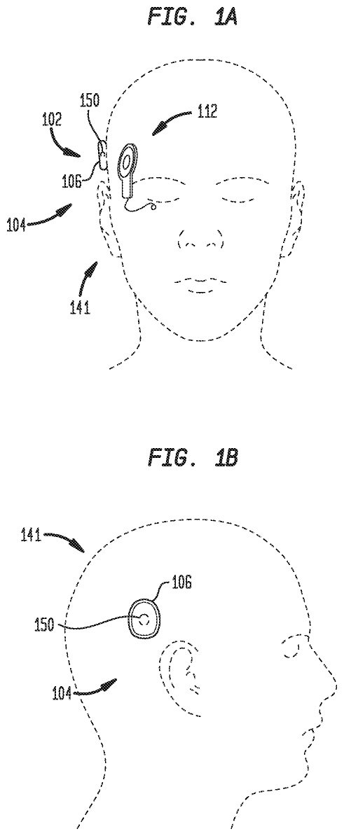

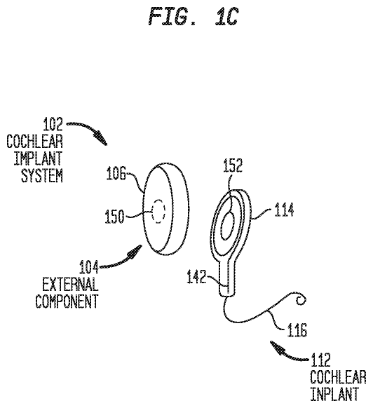

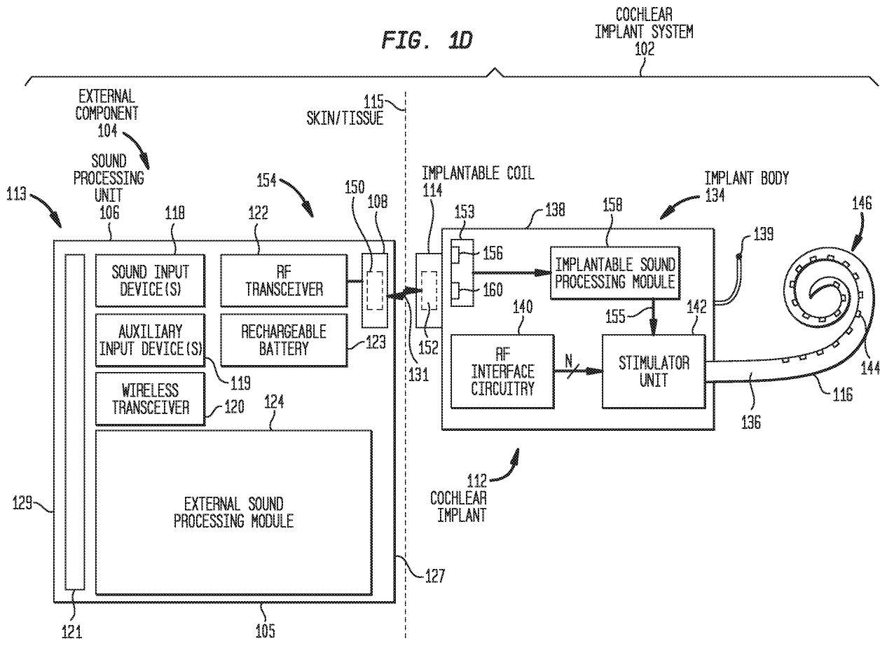

FIGS. 1A-1D illustrates an example cochlear implant system 102 configured to implement certain embodiments of the techniques presented herein. The cochlear implant system 102 comprises an external component 104 and an implantable component 112. In the examples of FIGS. 1A-1D, the implantable component is sometimes referred to as a “cochlear implant.” FIG. 1A illustrates the cochlear implant 112 implanted in the head 141 of a recipient, while FIG. 1B is a schematic drawing of the external component 104 worn on the head 141 of the recipient. FIG. 1C is another schematic view of the cochlear implant system 102, while FIG. 1D illustrates further details of the cochlear implant system 102. For ease of description, FIGS. 1A-1D will generally be described together.

As noted, cochlear implant system 102 includes an external component 104 that is configured to be directly or indirectly attached to the body of the recipient and an implantable component 112 configured to be implanted in the recipient. In the examples of FIGS. 1A-1D, the external component 104 comprises a sound processing unit 106, while the cochlear implant 112 includes an internal coil 114, an implant body 134, and an elongate stimulating assembly 116 configured to be implanted in the recipient's cochlea.

In the example of FIGS. 1A-1D, the sound processing unit 106 is an off-the-ear (OTE) sound processing unit, sometimes referred to herein as an OTE component, that is configured to send data and power to the implantable component 112. In general, an OTE sound processing unit is a component having a generally cylindrically shaped housing 105 and which is configured to be magnetically coupled to the recipient's head (e.g., includes an integrated external magnet 150 configured to be magnetically coupled to an implantable magnet 152 in the implantable component 112). The OTE sound processing unit 106 also includes an integrated external (headpiece) coil 108 that is configured to be inductively coupled to the implantable coil 114.

It is to be appreciated that the OTE sound processing unit 106 is merely illustrative of the external devices that could operate with implantable component 112. For example, in alternative examples, the external component may comprise a behind-the-ear (BTE) sound processing unit or a micro-BTE sound processing unit and a separate external. In general, a BTE sound processing unit comprises a housing that is shaped to be worn on the outer ear of the recipient and is connected to the separate external coil assembly via a cable, where the external coil assembly is configured to be magnetically and inductively coupled to the implantable coil 114. It is also to be appreciated that alternative external components could be located in the recipient's ear canal, worn on the body, etc.

As noted above, the cochlear implant system 102 includes the sound processing unit 106 and the cochlear implant 112. However, as described further below, the cochlear implant 112 can operate with the sound processing unit 106 stimulate the recipient or the cochlear implant 112 can operate independently from the sound processing unit 106, for at least a period, to stimulate the recipient. For example, the cochlear implant 112 can operate in a first general mode, sometimes referred to as an “external hearing mode,” in which the sound processing unit 106 captures sound signals which are then used as the basis for delivering stimulation signals to the recipient. The cochlear implant 112 can also operate in a second general mode, sometimes referred as an “invisible hearing” mode, in which the sound processing unit 106 is unable to provide sound signals to the cochlear implant 112 (e.g., the sound processing unit 106 is not present, the sound processing unit 106 is powered-off, the sound processing unit 106 is malfunctioning, etc.). As such, in the invisible hearing mode, the cochlear implant 112 captures sound signals itself via implantable sound sensors and then uses those sound signals as the basis for delivering stimulation signals to the recipient. Further details regarding operation of the cochlear implant 112 in the external hearing mode are provided below, followed by details regarding operation of the cochlear implant 112 in the invisible hearing mode. It is to be appreciated that reference to the external hearing mode and the invisible hearing mode is merely illustrative and that the cochlear implant 112 could also operate in alternative modes.

Referring first to the external hearing mode, FIGS. 1A-1D illustrate that the OTE sound processing unit 106 comprises one or more input devices 113 that are configured to receive input signals (e.g., sound or data signals). The one or more input devices 113 include one or more sound input devices 118 (e.g., one or more external microphones, audio input ports, telecoils, etc.), one or more auxiliary input devices 119 (e.g., audio ports, such as a Direct Audio Input (DAI), data ports, such as a Universal Serial Bus (USB) port, cable port, etc.), and a wireless transmitter/receiver (transceiver) 120. However, it is to be appreciated that one or more input devices 113 may include additional types of input devices and/or less input devices (e.g., the wireless short range radio transceiver 120 and/or one or more auxiliary input devices 119 could be omitted).

The OTE sound processing unit 106 also comprises the external coil 108, a charging coil 121, a closely-coupled transmitter/receiver (RF transceiver) 122, sometimes referred to as or radio-frequency (RF) transceiver 122, at least one rechargeable battery 123, and an external sound processing module 124. The external sound processing module 124 may comprise, for example, one or more processors and a memory device (memory) that includes sound processing logic. The memory device may comprise any one or more of: Non-Volatile Memory (NVM), Ferroelectric Random Access Memory (FRAM), read only memory (ROM), random access memory (RAM), magnetic disk storage media devices, optical storage media devices, flash memory devices, electrical, optical, or other physical/tangible memory storage devices. The one or more processors are, for example, microprocessors or microcontrollers that execute instructions for the sound processing logic stored in memory device.

The implantable component 112 comprises an implant body (main module) 134, a lead region 136, and the intra-cochlear stimulating assembly 116, all configured to be implanted under the skin/tissue (tissue) 115 of the recipient. The implant body 134 generally comprises a hermetically-sealed housing 138 in which RF interface circuitry 140 and a stimulator unit 142 are disposed. The implant body 134 also includes the internal/implantable coil 114 that is generally external to the housing 138, but which is connected to the transceiver 140 via a hermetic feedthrough (not shown in FIG. 1D).

As noted, stimulating assembly 116 is configured to be at least partially implanted in the recipient's cochlea. Stimulating assembly 116 includes a plurality of longitudinally spaced intra-cochlear electrical stimulating contacts (electrodes) 144 that collectively form a contact or electrode array 146 for delivery of electrical stimulation (current) to the recipient's cochlea.

Stimulating assembly 116 extends through an opening in the recipient's cochlea (e.g., cochleostomy, the round window, etc.) and has a proximal end connected to stimulator unit 142 via lead region 136 and a hermetic feedthrough (not shown in FIG. 1D). Lead region 136 includes a plurality of conductors (wires) that electrically couple the electrodes 144 to the stimulator unit 142. The implantable component 112 also includes an electrode outside of the cochlea, sometimes referred to as the extra-cochlear electrode (ECE) 139.

As noted, the cochlear implant system 102 includes the external coil 108 and the implantable coil 114. The external magnet 152 is fixed relative to the external coil 108 and the implantable magnet 152 is fixed relative to the implantable coil 114. The magnets fixed relative to the external coil 108 and the implantable coil 114 facilitate the operational alignment of the external coil 108 with the implantable coil 114. This operational alignment of the coils enables the external component 104 to transmit data and power to the implantable component 112 via a closely-coupled wireless RF link 131 formed between the external coil 108 with the implantable coil 114. In certain examples, the closely-coupled wireless link 131 is a radio frequency (RF) link. However, various other types of energy transfer, such as infrared (IR), electromagnetic, capacitive and inductive transfer, may be used to transfer the power and/or data from an external component to an implantable component and, as such, FIG. 1D illustrates only one example arrangement.

As noted above, sound processing unit 106 includes the external sound processing module 124. The external sound processing module 124 is configured to convert received input signals (received at one or more of the input devices 113) into output signals for use in stimulating a first ear of a recipient (i.e., the external sound processing module 124 is configured to perform sound processing on input signals received at the sound processing unit 106). Stated differently, the one or more processors in the external sound processing module 124 are configured to execute sound processing logic in memory to convert the received input signals into output signals that represent electrical stimulation for delivery to the recipient.

As noted, FIG. 1D illustrates an embodiment in which the external sound processing module 124 in the sound processing unit 106 generates the output signals. In an alternative embodiment, the sound processing unit 106 can send less processed information (e.g., audio data) to the implantable component 112 and the sound processing operations (e.g., conversion of sounds to output signals) can be performed by a processor within the implantable component 112.

Returning to the specific example of FIG. 1D, the output signals are provided to the RF transceiver 122, which transcutaneously transfers the output signals (e.g., in an encoded manner) to the implantable component 112 via external coil 108 and implantable coil 114. That is, the output signals are received at the RF interface circuitry 140 via implantable coil 114 and provided to the stimulator unit 142. The stimulator unit 142 is configured to utilize the output signals to generate electrical stimulation signals (e.g., current signals) for delivery to the recipient's cochlea. In this way, cochlear implant system 102 electrically stimulates the recipient's auditory nerve cells, bypassing absent or defective hair cells that normally transduce acoustic vibrations into neural activity, in a manner that causes the recipient to perceive one or more components of the received sound signals.

As detailed above, in the external hearing mode the cochlear implant 112 receives processed sound signals from the sound processing unit 106. However, in the invisible hearing mode, the cochlear implant 112 is configured to capture and process sound signals for use in electrically stimulating the recipient's auditory nerve cells. In particular, as shown in FIG. 1D, the cochlear implant 112 includes a plurality of implantable sensors 153 and an implantable sound processing module 158. Similar to the external sound processing module 124, the implantable sound processing module 158 may comprise, for example, one or more processors and a memory device (memory) that includes sound processing logic. The memory device may comprise any one or more of: Non-Volatile Memory (NVM), Ferroelectric Random Access Memory (FRAM), read only memory (ROM), random access memory (RAM), magnetic disk storage media devices, optical storage media devices, flash memory devices, electrical, optical, or other physical/tangible memory storage devices. The one or more processors are, for example, microprocessors or microcontrollers that execute instructions for the sound processing logic stored in memory device.

In the invisible hearing mode, the implantable sensors 153 are configured to detect/capture signals (e.g., acoustic sound signals, vibrations, etc.), which are provided to the implantable sound processing module 158. The implantable sound processing module 158 is configured to convert received input signals (received at one or more of the implantable sensors 153) into output signals for use in stimulating the first ear of a recipient (i.e., the processing module 158 is configured to perform sound processing operations). Stated differently, the one or more processors in implantable sound processing module 158 are configured to execute sound processing logic in memory to convert the received input signals into output signals 155 that are provided to the stimulator unit 142. The stimulator unit 142 is configured to utilize the output signals 155 to generate electrical stimulation signals (e.g., current signals) for delivery to the recipient's cochlea, thereby bypassing the absent or defective hair cells that normally transduce acoustic vibrations into neural activity.

It is to be appreciated that the above description of the so-called external hearing mode and the so-called invisible hearing mode are merely illustrative and that the cochlear implant system 102 could operate differently in different embodiments. For example, in one alternative implementation of the external hearing mode, the cochlear implant 112 could use signals captured by the sound input devices 118 and the implantable sensors 153 in generating stimulation signals for delivery to the recipient.

As noted above, the cochlear implant 112 comprises implantable sensors 153. In certain embodiments, the implantable sensors 153 comprise at least two sensors 156 and 160, where at least one of the sensors is designed to be more sensitive to bone-transmitted vibrations than it is to acoustic (air-borne) sound waves. In the illustrative embodiment of FIG. 1D, the implantable sensor 156 is an implantable “sound” sensor/transducer that is primarily configured to detect/receive external acoustic sounds (e.g., an implantable microphone), while the implantable sensor 160 is a “vibration” sensor that is primarily configured to detect/receive internal vibration signals, including body noises. (e.g., another implantable microphone or an accelerometer). These sensors can take a variety of different forms, such as another implantable microphone, an accelerometer, etc. However, for ease of description, embodiments presented herein will be primarily described with reference to the use of an implantable microphone as the sound sensor and an accelerometer as the vibration sensor. The increased sensitivity of the accelerometer to vibration signals (e.g., body noises) may be due to, for example, the structure of the accelerometer relative to the microphone, the implanted position of the accelerometer relative to the microphone, etc. For example, in certain embodiments, the accelerometer and the microphone are structurally similar but they are placed in different locations which accounts for the vibration/body noise sensitivity difference. Again, it is to be appreciated that these specific implementations are non-limiting and that embodiments of the present invention may be used with different types of implantable sensors

The implantable microphone 156 and the accelerometer 160 can each be disposed in, or electrically connected to, the implant body 134. In operation, the implantable microphone 156 and the accelerometer 160 each detect input signals and convert the detected input signals into electrical signals. The input signals detected by the implantable microphone 156 and the accelerometer 160 can each include external acoustic sounds and/or vibration signals, including body noises.

Implantable sensors, when positioned in the body of a recipient, generally have a different response to input signals than an external sensor positioned outside of the body of the recipient. For example, an implanted subcutaneous microphone (e.g., an implantable microphone implanted in the body of the recipient) may have a different frequency response than a substantially similar microphone positioned outside of the recipient. In particular, the sensitivity to acoustic inputs at each frequency of an implanted subcutaneous microphone is influenced by many factors that are difficult to predict and/or measure, such as skin flap thickness, coupling to underlying bone, implant location in the head, etc. As such, there is a need to “equalize” the sensitivity to acoustic inputs (at each frequency) of an implantable sound sensor (e.g., implanted subcutaneous microphone) to that of a similar external sound sensor (e.g., external microphone). As used herein, “equalizing” the sensitivity to acoustic inputs of an implantable sound sensor refers to application of an equalization filter to the signals detected by the implantable sound sensor, where the equalization filter coefficients are selected so to make the sensitivity to acoustic inputs at each frequency of the implantable sound signals substantially the same as the sensitivity to acoustic inputs at each frequency of the sound signals generated by the similar external sound sensor.

As described below, presented herein are techniques to equalize the sensitivity to acoustic inputs of an implantable sound sensor to that of a similar external sound sensor based on a coherence between the signals captured by the implantable sound sensor and the signals captured by the external sound sensor. In accordance with certain embodiments presented herein, the filter coefficients are dynamically (e.g., adaptively) updated during normal use (e.g., based on signals received during normal operation of the cochlear implant 112) and the coherence is used to control the rate of change or speed at which the filter coefficients are dynamically updated.

As noted, implantable sensors, when positioned in the body of the recipient, are subject to unwanted vibration, sometimes referred to as “body-noises” (e.g., the will capture vibrations of the body). Although some implantable sensors are intended to capture such vibration signals, other sensors, such as implantable microphones, are not. In general, the unwanted vibrations (body noises) can be attenuated through use of a second transducer designed as a pure vibration sensor and a filtering process to remove vibration-based bone conducted sounds. This filtering process is sometimes referred to herein as a vibration/body noise filter or body noise canceller. Stated differently, for implantable sensors that are not intended to capture vibration signals, a body noise cancellation filter (body noise canceller) is applied to the implantable sound signals in order to substantially remove the vibration signals, which include body noises. In the case of an implantable microphone, the removal of the vibration signals leaves substantially only the desired acoustic sound signals.

In addition to body-generated vibration, the use of a contralateral hearing aid with sufficiently high amplification can induce vibration in the head. During the measurement of acoustic sensitivity, this acoustically induced vibration can dominate the response of the microphone at some frequencies, such that the vibration induced response is larger than the direct acoustic response. In normal operation of the device, the body noise canceller successfully removes the induced vibration component, leaving only the direct acoustic component. Therefore, it is good practice to equalize the response after the body noise canceller has removed the induced vibration component from the signal.

As such, also presented herein are techniques that calibrate a body noise filter during normal use (e.g., based on signals received during normal operation of the cochlear implant 112). The techniques presented herein calibrate (update) the body noise filter coefficients based on a coherence of (between) the signals detected by the implantable sound sensor and the implantable vibration signals generated by the implantable vibration sensor. Specifically, the favorable conditions for updating the body noise filter is when only or primarily vibration signals (body noise) are present in the signals detected by the implantable sound sensor and the signals detected by the implantable vibration sensor, as indicated by the coherence between the detected signals. In accordance with certain embodiments presented herein, the coherence between the signals detected by the implantable sound sensor and the signals detected by the implantable vibration sensor is used to control the rate of change or speed at which the body noise filter coefficients are dynamically updated.

As described further below, the cochlear implant 112 is also configured to automatically detect favorable acoustic conditions under which the equalization filter coefficients can be updated. Specifically, the favorable conditions for updating the equalization filter is when only or primarily acoustic signals are present in the signals detected by the implantable sound sensor and the signals detected by the external sound sensor, as indicated by the coherence between the detected signals. In accordance with certain embodiments presented herein, the coherence between the signals detected by the implantable sound sensor and the signals detected by the external sound sensor is used to control the rate of change or speed at which the equalization filter coefficients are dynamically updated.

For both the body noise filter and the equalization filter, the detection mechanism uses magnitude squared coherence as the principal indicator of favorable conditions. This means that the system will update the coefficients of the body noise filter and/or the coefficients of the equalization filter when the corresponding magnitude squared coherence (e.g., the coherence between the signals detected by the implantable sound sensor and the signals detected by the external sound sensor for the equalization filter or the coherence between the signals detected by the implantable sound sensor and the signals detected by the implantable vibration sensor for the bod noise filter) is acceptable during general use of the device.

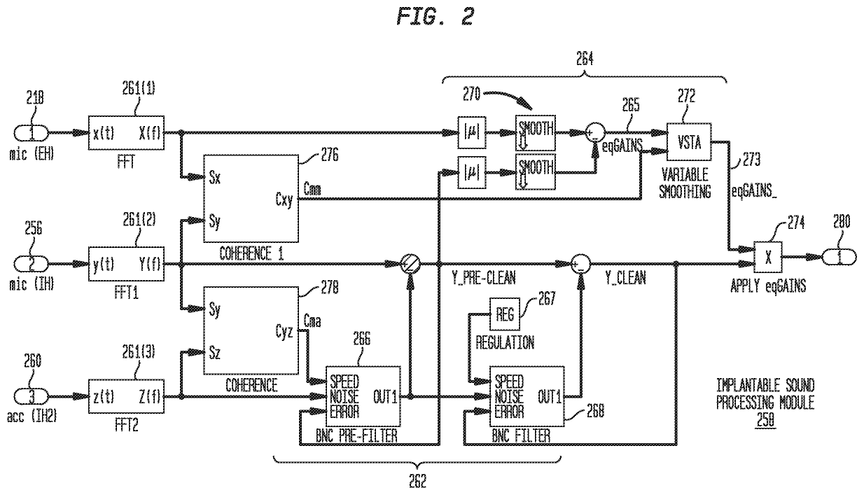

FIG. 2 is a schematic block diagram illustrating operation of an implantable sound processing module of a cochlear implant, such as cochlear implant 112, in accordance with embodiments. For ease of description, the implantable sound processing module of FIG. 2 is referred to as implantable sound processing module 258.

As shown in FIG. 2, the implantable sound processing module 258 is configured to receive input signals from three (3) sensors, including an external microphone (EH) 218 generating input signals x (t), an implantable microphone (IH) 256 generating signals y(t), and an implantable accelerometer (IH2) 260 generating signals z(t). Fast Fourier Transforms (FFTs) 261(1), 261(2), and 261(3) are applied to the signals x(t), y(t), and z(t), respectively, to convert the signals to a representation in the frequency domain. As such, after the FFTs 261, the input signals detected by the external microphone 218 are referred to as X(f), the input signals detected by the implantable microphone 256 are referred to as Y(f), and the input signals detected by the implantable accelerometer 260 are referred to as Z(f).

The implantable sound processing module 258 generally comprises two primary filtering sub-modules. The first filtering sub-module in the implantable sound processing module 258 is referred to as the body noise canceller 262. The body noise canceller 262 is used for attenuation of vibration components (body noises) in the input signals Y(f) detected by the implantable microphone. In the example of FIG. 2, the body noise canceller 262 generally comprises two parts/stages, referred to as the body noise canceller (BNC) pre-filter 266 (first body noise cancellation filter) and the body noise filter 268 (second body noise cancellation filter).

The second filtering sub-module in the implantable sound processing module 258 is referred to as the equalization module 264. The equalization module 264 is used to equalize the magnitude response of the internal microphone 256 to that of the external microphone 218 based on a coherence between the signals X(f) detected by the external microphone 218 and the signals Y(f) detected by the implantable microphone 256. The equalization module 264 comprises an equalization gain calculation block 270, variable smoothing block 272, and an equalization filter 274. Further details regarding operation of the equalization module 264 are provided below.

As described further below, the techniques presented herein utilize a measure referred to as the “magnitude squared coherence” to control the adaptation of the body noise canceller 262 and the equalization module 264 so that they adapt under favorable acoustic/vibration conditions. In general, magnitude squared coherence provides a frequency domain measure of how well two signals are correlated with one another. As detailed below, the magnitude squared coherence is calculated from the time-averaged auto and cross correlation power spectrums of the two signals, where the power spectrums are smoothed over time before the coherence is calculated. The coherence at each frequency is a value between zero (0) and one (1), where one represents high coherence and zero represents no coherence as would be the case with two uncorrelated noise signals.

The calculated coherence value is used to control the update of the body noise canceller 262 and the equalization module 264, more specifically the speed/rate at which the equalization filter coefficients and the body noise filter coefficients are dynamically updated (e.g., adapted). When the coherence is high, the corresponding filter coefficients are updated more quickly. However, when the coherence is low, the corresponding filter coefficients are updated more slowly. The smoothing can be chosen to provide quite slow update for general use, over minutes, hours, or even days. The smoothing can be adjusted to update faster under conditions where the acoustic environment is favorable, such as the user initiating an equalization measurement, or the clinician making a measurement in the clinic.

Shown in FIG. 2 is an equalization coherence block 276 and a body noise coherence block 278 that are used to calculate the magnitude squared coherence of (between) two input signals provided to the corresponding block. The equalization coherence block 276 calculates the coherence between the signals X(f) detected by the external microphone 218 and the signals Y(f) detected by the implantable microphone 256), and generates a coherence signal C_{MM}. The body noise coherence block 278 calculates the coherence between the signals Z(f) detected by the implantable accelerometer and the signals Y(f) detected by the implantable microphone, and generates a coherence signal C_{MA}.

As noted, the equalization module 264 first includes the equalization gain calculation block 270. The equalization gain calculation block 270 is configured to determine, in real-time, equalization filter coefficients 265 (eqGains) from the signals X(f) and the signals Y(f). The equalization module 264 further includes the variable smoothing block 272 that stores previously determined equalization filter coefficients.

The variable smoothing block 272 receives the real-time equalization filter coefficients 265 from the equalization gain calculation block 270, as well as the coherence signal C_{MM} generated from the signals X(f) detected by the external microphone 218 and the signals Y(f) detected by the implantable microphone 256. At the variable smoothing block 272, the coherence signal C_{MM} is used to control how the previously determined equalization filter coefficients stored in the variable smoothing block 272 are dynamically updated to match (i.e., adjusted towards) the real-time determined equalization filter coefficients 265. In other words, in certain embodiments, the variable smoothing block 272 is a first order smoothing block where a sequence of estimates are smoothed over time, where the coherence (C_{MM}) controls the smoothing time (e.g., C_{MM} controls the rate of change of the previously determined equalization filter coefficients).

When the coherence signal C_{MM} is relatively high, the previously determined equalization filter coefficients are updated more quickly to move towards the real-time determined equalization filter coefficients 265. However, when the coherence signal C_{MM} is low, the previously determined equalization filter coefficients are updated more slowly. Stated differently, if the coherence is high, the smoothing time constant is increased to enable faster updating, while when the coherence is low, the time constant is made very small to prevent or limit the updating. The output of the variable smoothing block 272 are the updated equalization filter coefficients (eqGains_) 273 that are used by the equalization filter 274 to equalize (filter) the implantable microphone signal Y(f).

The coherence signal C_{MM} can be used in a number of different manners to control the rate of change of the previously determined equalization filter coefficients. As noted, the coherence is a value between 0 and 1 and the rate of change of the coefficients can be an adjustable value (e.g., a sliding scaled value), with an increasingly high rate of change as the coherence approaches a value of 1 and an increasingly lower rate of change as the coherence approaches a value of 0.

In certain embodiments, one of more thresholds may be introduced to limit a rate of change of the equalization filter coefficients for a given coherence. For example, if the coherence signal C_{MM} is below a predetermined threshold, the variable smoothing block 272 can be configured to prevent updating of the previously determined equalization filter coefficients.

As noted, the coherence signal C_{MM} is generated from the signals X(f) detected by the external microphone 218 and the signals Y(f) detected by the implantable microphone 256. However, also as noted above, a cochlear implant, such as cochlear implant 112 or another implantable component, can operate for periods of time without an external component and, as such, without receiving signals X(f) detected by the external microphone 218. In such circumstances, the coherence signal C_{MM} would be 0 or a very low value and, accordingly, the previously determined equalization filter coefficients are not updated

In summary of the above, the equalization module 264 operates to determine equalization filter coefficients (equalization gains) that, when applied to the signals Y(f) detected by the implantable microphone 256, equalize the sensitivity to acoustic inputs to that of the external microphone 218. As noted, the variable smoothing block 272 is introduced to control the update speed at which the equalization filter coefficients are adapted, where the update is controlled by the coherence C_{MM}. When the coherence C_{MM} is low, or the signals from the external microphone 218 are missing, the eqGains are not updated. However, when the coherence C_{MM} is high, the eqGains are updated more quickly (e.g., faster rate of change is allowed).

It is noted that the eqGains uses the output of the BNC pre-filter 266, and not the output of the BNC filter 268. In this way, the BNC filter 268 can operate normally, providing body noise reduction for the recipient, while the calibration blocks can continue to operate, using a stable yet substantially body noise free signal.

As noted above, the body noise canceller 262 includes the BNC pre-filter 266 and the BNC filter 264. In certain embodiments, the BNC filter 264 is an adaptive Normalized least mean squares (NLMS) filter in the frequency domain. The speed of adaptation of the BNC filter 264 is controlled by the parameter setting of the regulation block 267. In an alternative embodiment, the speed of adaptation of the BNC filter 264 could also be regulated based on the coherence C_{MA}.

The BNC pre-filter 266 is, in certain embodiments, an adaptive NLMS filter, with a similar structure as that of the BNC filter 264. However, the BNC pre-filter 266 operates as a calibration filter that is essentially fixed and updated only when the conditions are favorable. That is, the coherence signal C_{MA} determined by the body noise coherence block 278 controls the update speed at which the filter coefficients/gains of the BNC pre-filter 266 are adapted. When the coherence signal C_{MA} is low, the filter coefficients/gains of the BNC pre-filter are not updated or are updated more slowly. However, when the coherence signal C_{MA} is high, the filter coefficients/gains of the BNC pre-filter are updated more quickly (e.g., faster rate of change is allowed).

As noted, the body noise reduction is split into two parts, the BNC pre-filter 266 and the BNC filter 264. In the example arrangement of FIG. 6, the BNC pre-filter 266 is configured to attenuate a majority of the body noise and is essentially fixed, providing a stable signal from which to calculate the equalization gains, as described below. The BNC filter 264 provides additional noise cancellation when the system changes dynamically.

In FIG. 2, the implantable sound processing module 258 generates an output signal 280. The output signal 280 is a processed form of the signals Y(f) detected by the implantable microphone 256, filtered by the body noise canceller 262 and the equalization module 264 (i.e., to which the body noise filter and equalization gains have bene applied thereto).

In certain embodiments, the BNC pre-filter 266 is a complex transfer function and the coefficients of the BNC pre-filter 266 include both magnitude/amplitude and phase components that are each updated based on the coherence signal C_{MA}. That is, both a magnitude and phase of the coefficients of the BNC pre-filter 266 (first body noise cancellation filter) are adapted based on the coherence between the signals Y(f) detected by the implantable microphone 256 and the signals Z(f) detected by the implantable accelerometer 260. In contrast, the coefficients of the equalization filter can include only the magnitude/amplitude components that are updated based on the coherence signal C_{MM}. That is, only a magnitude of the coefficients of the equalization filter are updated based on the coherence between the signals X(f) detected by the external microphone 218 and the signals Y(f) detected by the implantable microphone 256.

As noted above, the BNC filter coefficients are updated when vibration input is dominant, while the equalization filter coefficients are updated when an acoustic input is dominant. As a result, the two coherence measures C_{MM} and C_{MA} could be used to inhibit one another. Therefore, in certain examples, only one of the two filters are updated at a given time. This mix may vary across frequency. For example, in one illustrative arrangement, the following rules could be applied:
If C_{MM} is high and C_{MM}>C_{MA}, update the eqGains filter
If C_{MA} is high and C_{MA}>C_{MM}, update the BNC filter

As noted, the techniques presented herein utilize the magnitude squared coherence to control the rate of change of the filter coefficients for each of the equalization module 264 and the BNC pre-filter 266. The magnitude squared coherence is calculated as shown below in Equation 1.

$${C}_{x y} [ k , n ] = \frac{{\vert [ k , n ] \vert}^{2}}{[ k , n ] [ k , n ]} \qquad Equation 1$$

The coherence can be optionally thresholded to completely prevent adaptation when the coherence is low, as shown below in Equation 2.

$${C}_{x y} [ k , n ] = \{ \begin{matrix}0 , {C}_{x y} [ k , n ] < {C}_{thresh} [ k ] \\ {C}_{x y} [ k , n ] otherwise\end{matrix} \qquad Equation 2$$

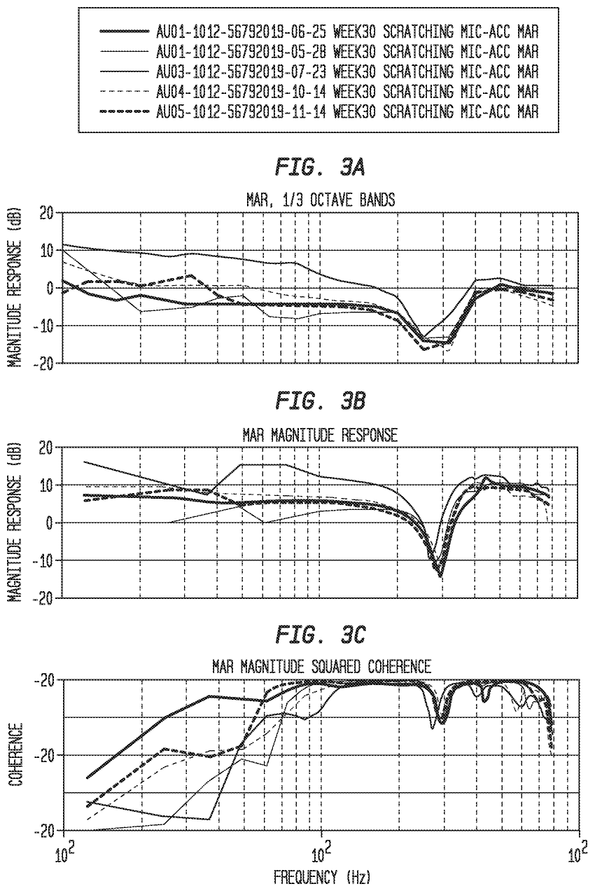

FIGS. 3A, 3B, and 3C generally illustrate example inputs for five (5) recipients in which the coherence of (between) the signals Z(f) detected by the implantable accelerometer 260 and the signals Y(f) detected by the implantable microphone 256 is relatively high, resulting in an increase in the rate at which the filter coefficients for the BNC pre-filter 266 are updated. More specifically, each of FIGS. 3A, 3B, and 3C, include five lines/traces corresponding to five different recipients with a scratching noise vibration input. As shown, the coherence is high across a broad frequency range, indicating favorable conditions for updating the BNC pre-filter 266.

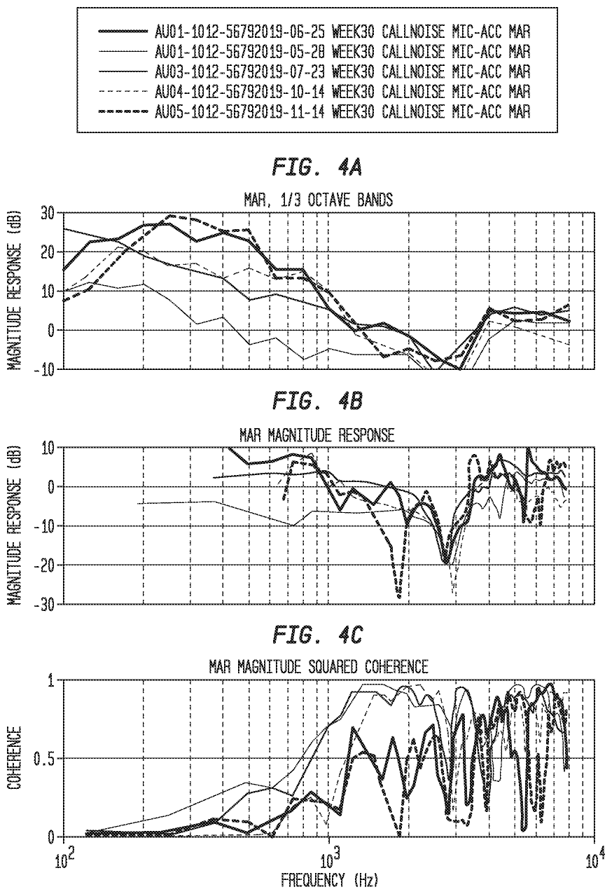

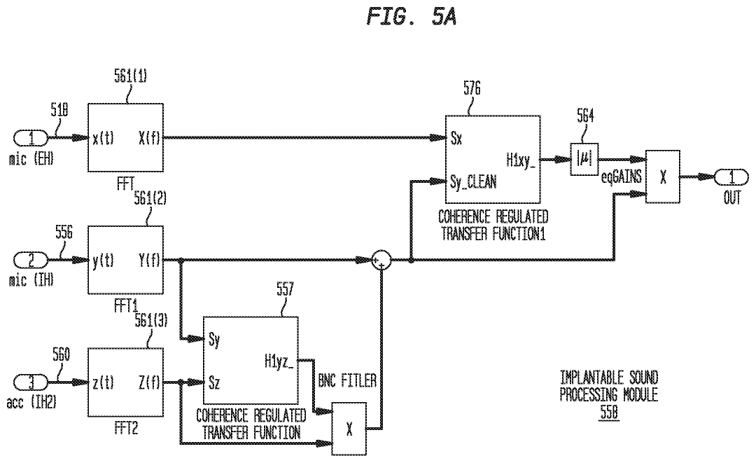

In contrast to FIGS. 3A, 3B, and 3C, FIGS. 4A, 4B, and 4C illustrate example inputs for five (5) recipients in which the coherence of (between) the signals Z(f) detected by the implantable accelerometer 260 and the signals Y(f) detected by the implantable microphone 256 is relatively low, resulting in a decrease in the rate at which the filter coefficients for the BNC pre-filter 266 are updated. More specifically, each of FIGS. 4A, 4B, and 4C, include five lines/traces corresponding to five different recipients with an acoustic input. As shown, the coherence is low across a broad frequency range, indicating unfavorable conditions for updating the BNC pre-filter 266.

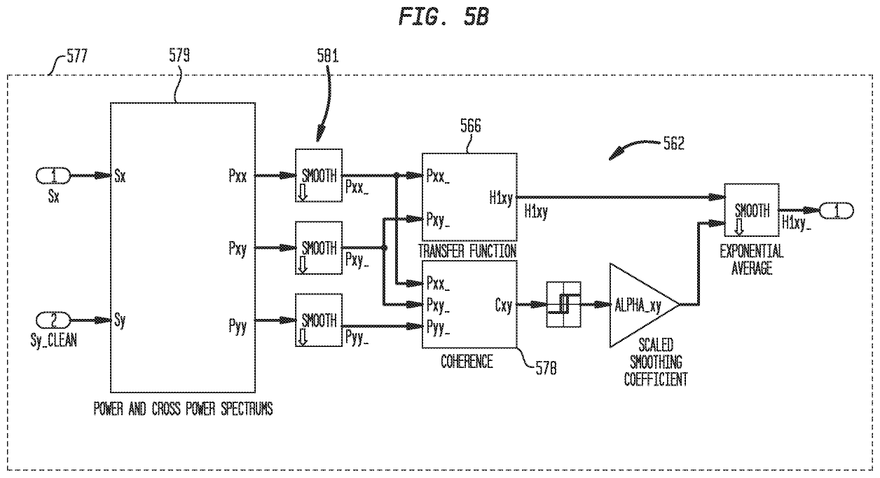

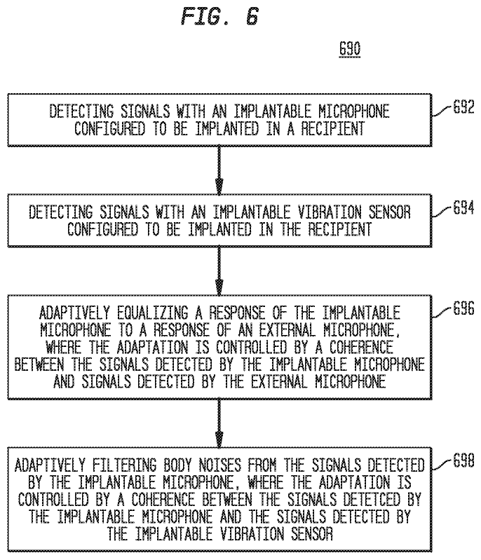

As noted, FIG. 2 generally illustrates one example of an implantable sound processing module of an implantable component in which the BNC pre-filter is updated using an adaptive feedback loop. FIGS. 5A and 5B illustrate an alternative embodiment in which the BNC pre-filter is updated using a direct calculation (e.g., feed forward implementation).

More specifically, FIG. 5A is a schematic block diagram illustrating operation of an implantable sound processing module of a cochlear implant, such as cochlear implant 112, in accordance with embodiments. For ease of description, the implantable sound processing module of FIG. 5A is referred to as implantable sound processing module 558.

In the embodiment of FIG. 5A, body noise cancellation is performed at block 557. The operations of block 557 are shown in greater detail in FIG. 5B. For ease of description, FIGS. 5A and 5B will be described together.

As shown in FIG. 5A, the implantable sound processing module 558 is configured to receive input signals from three (3) sensors, including an external microphone (EH) 518 generating input signal x(t), an implantable microphone (IH) 556 generating input signal y(t), and an implantable accelerometer (IH2) 560 generating input signal z(t). Fast Fourier Transforms (FFTs) 561(1), 561(2), and 251(3) are applied to the signals x(t), y(t), and z(t), respectively, to convert the signals to a representation in the frequency domain. As such, after the FFTs 561, the input signal from the external microphone 518 is referred to as X(f), the input signal from the implantable microphone 556 is referred to as Y(f), and the input signal from the implantable accelerometer 560 is referred to as Z(f).

The implantable sound processing module 558 generally comprises two primary filtering modules. The first filtering module in the implantable sound processing module 558 is referred to as the body noise canceller 562 (FIG. 5B), which is used for attenuation of vibration components (body noises) in the implantable microphone input signal Y(f). The second filtering module in the implantable sound processing module 558 is referred to as the equalization module 564. The equalization module 564 is used to equalize the equalize the sensitivity to acoustic inputs of the implantable microphone 556 to that of the external microphone 518, and is generally implemented as described above with reference to equalization module 264 of FIG. 2. The output 580 has the body noise canceller and equalization gains applied thereto.

As described above, the techniques presented herein utilize the magnitude squared coherence to control the adaptation of the body noise canceller 562 and the equalization module 564 so that they adapt under favorable acoustic/vibration conditions, more specifically the speed/rate at which the coefficients of the two filters adapt/update. When the coherence is high, the corresponding filter coefficients are updated more quickly. However, when the coherence is low, the corresponding filter coefficients are updated more slowly. The smoothing can be chosen to provide quite slow update for general use, over minutes, hours, or even days. The smoothing can be adjusted to update faster under conditions where the acoustic environment is favorable, such as the user initiating an equalization measurement, or the clinician making a measurement in the clinic.

Shown in FIG. 5A is an equalization coherence block 576, which calculates the coherence between the signals X(f) detected by the external microphone 518 and the signals Y(f) detected by the implantable microphone 556. The equalization coherence block 576 generates a coherence signal C_{MM}. Shown in FIG. 5B is a body noise coherence block 578 that are used to calculate the magnitude squared coherence between the signals Z(f) detected by the implantable accelerometer signal 560 and the signals Y(f) detected by the implantable microphone 556. The body noise coherence block 578 generates a coherence signal C_{MA}.

In FIG. 5A, the eqGains are calculated using the transfer function between X and Y_clean, which is used to equalize the response of Y_clean to be equal to the external microphone. The eqGains are updated during favorable conditions when the coherence between X and Y_clean (C_{MM}) is high. This occurs when acoustic-based inputs dominate. Note that the coherence calculation for regulating the update of the equalization filter coefficients could also be based on the coherence between X and Y, rather than X and Y_clean. Regardless, the calculation of the transfer function is between X and Y_clean in order to calculate the eqGains correctly.

As noted, FIGS. 5A and 5B illustrate a direct calculation (e.g., feed forward implementation) of the body noise filter 562. As noted, the input signals x(t), y(t) and z(t) are windowed and transformed to the frequency domain with FFTs, to create the complex frequency domain representations X(k,n), Y(k,n) and Z(k,n), with frequency bin k and time frame index n. The BNC filter is calculated using the coherence regulated transfer function updated under favorable conditions when the coherence between Y and Z (C_{MA}) is high. This occurs when vibration-based inputs dominate the input signal. The filter is applied to Z and the resulted subtracted from Y to create the clean output Y_clean, which has vibration removed.

In particular, at block 577, the power and cross-power spectrums of Y(k,n) and Z(k,n) are calculated at block 579. In general, the auto-power spectrums are calculated as shown in Equations 3 and 4, where * indicates complex conjugate.

Pₓₓ[k,n]=X*[k,n]X[k,n] Equation 3

P_{yy}[k,n]=Y*[k,n]Y[k,n] Equation 4

And the cross-power spectrum is calculated as shown below in Equation 5.

P_{xy}[k,n]=X*[k,n]Y[k,n] Equation 5

As shown in FIG. 5B, at 581, the auto and cross power spectrums are exponentially smoothed using first order IIR filters with smooth coefficient α, which is chosen to ensure that the coherence and transfer function estimates are stable (smoothing over a few seconds). The smoothing operations are defined as shown below in Equations 6, 7, and 8.

[symbol]

[k,n]=αPₓₓ[k,n]+(1−α) [symbol] [k,n−1] Equation 6

[symbol]

[k,n]=αP_{yy}[k,n]+(1−α) [symbol] [k,n−1] Equation 7

[symbol]

[k,n]=αP_{xy}[k,n]+(1−α) [symbol] [k,n−1] Equation 8

In FIG. 5B, the BNC filter coefficients are calculated at 566, as shown below in Equation 9. As shown, the transfer function is calculated as the ratio of the smoothed cross-power of Equation 8 and the smoothed power spectrum of Equation 6.

$${H}_{x y} [ k , n ] = \frac{[ k , n ]}{[ k , n ]} \qquad Equation 9$$

In FIG. 5B, the magnitude squared coherence is calculated at 578, as shown below in Equation 10.

$${C}_{x y} [ kn ] = \frac{{\vert [ k , n ] \vert}^{2}}{[ k , n ] [ k , n ]} \qquad Equation 10$$

In certain embodiments, the coherence can be optionally thresholded to completely prevent adaptation when the coherence is low, as shown below in Equation 11.

$${C}_{x y} [ k , n ] = \{ \begin{matrix}0 , {C}_{x y} [ k , n ] < {C}_{thresh} [ k ] \\ {C}_{x y} [ k , n ] otherwise\end{matrix} \qquad Equation 1 l$$

Finally, the transfer function is smoothed using first order IIR filter 579, where the smoothing coefficient, β is scaled by the coherence, such that the filter is updated only when the coherence is high. The smoothing coefficient β is chosen so that the filter is updated quite slowly, over minutes, hours, or even days. The smoothing can be adjusted to update faster under conditions where the acoustic environment is favorable, such as the user initiating a measurement, or the clinician initiating a measurement in the clinic, as shown below in Equation 12.

$$[ k , n ] = β {C}_{x y} [ k , n ] {H}_{x y} [ k , n ] + ( 1 - β {C}_{x y} [ k , n ] ) [ k , n - 1 ] \qquad Equation 12$$

FIG. 6 is a flowchart of an example method 690, in accordance with certain embodiments presented. Method 690 begins at 692 where an implantable microphone, which is configured to be implanted in a recipient, detects signals. At 694, an implantable vibration sensor, which is also configured to be implanted in the recipient, detects signals. At 696, a response of the implantable microphone is adaptively equalized to a response of an external microphone, where the adaptation is controlled by a coherence between the signals detected by the implantable microphone and signals detected by the external microphone. At 696, vibration signals, including bode noises, are adaptively filtered from the signals detected by the implantable microphone based on a coherence between the implantable microphone signals and the body noise signals, where the adaptation is controlled by a coherence between the signals detected by the implantable microphone and the signals detected by the implantable vibration sensor.

As detailed above, the techniques presented here are generally directed to setting the coefficients (gains) of two filters, namely the body noise filter and the microphone equalization filter. The techniques presented herein are configured to determine favorable conditions for setting each of the filters based on a coherence between relevant signals, which allows the body noise filter and the microphone equalization filter to dynamically update on the fly during normal operation. The techniques presented may provide an automatic and reliable microphone equalization procedure that requires no intervention from the user or clinician. Options are provided to allow a semi-automatic measurement under loosely controlled acoustic conditions such as provided a stimulus from a smart phone, and to revert to a fully controlled acoustic measurement under calibrated conditions in the sound booth, as is the current clinical practice.

As noted elsewhere herein, embodiments presented herein have been primarily described with reference to an example auditory prosthesis system, namely a cochlear implant system. However, as noted above, it is to be appreciated that the techniques presented herein may be implemented by a variety of other types of implantable medical devices (or systems that include other types of implantable medical devices). For example, the techniques presented herein may be implemented by other auditory prostheses, such as acoustic hearing aids, middle ear auditory prostheses, bone conduction devices, direct acoustic stimulators, electro-acoustic prostheses, other electrically simulating auditory prostheses (e.g., auditory brain stimulators), etc. The techniques presented herein may also be implemented by tinnitus therapy devices, vestibular devices (e.g., vestibular implants), visual devices (i.e., bionic eyes), sensors, pacemakers, drug delivery systems, defibrillators, functional electrical stimulation devices, catheters, seizure devices (e.g., devices for monitoring and/or treating epileptic events), sleep apnea devices, electroporation devices, etc.

It is to be appreciated that the embodiments presented herein are not mutually exclusive and that the various embodiments may be combined with another in any of a number of different manners.

The invention described and claimed herein is not to be limited in scope by the specific preferred embodiments herein disclosed, since these embodiments are intended as illustrations, and not limitations, of several aspects of the invention. Any equivalent embodiments are intended to be within the scope of this invention. Indeed, various modifications of the invention in addition to those shown and described herein will become apparent to those skilled in the art from the foregoing description. Such modifications are also intended to fall within the scope of the appended claims.

1. A method, comprising:
detecting signals with an implantable microphone configured to be implanted in a recipient;
detecting signals with an implantable vibration sensor configured to be implanted in the recipient;
adaptively equalizing a response of the implantable microphone to a response of an external microphone, where the adaptation is controlled by a coherence between the signals detected by the implantable microphone and signals detected by the external microphone; and
adaptively filtering vibration signals from the signals detected by the implantable microphone, where the adaptation is controlled by a coherence between the signals detected by the implantable microphone and the signals detected by the implantable vibration sensor.

2. The method of claim 1, wherein detecting signals with an implantable vibration sensor comprises:
detecting vibration signals with an implantable accelerometer.

3. The method of claim 1, wherein detecting signals with an implantable vibration sensor comprises:
detecting vibration signals with a second implantable microphone.

4. The method of claim 1, wherein adaptively equalizing a response of the implantable microphone to a response of the external microphone comprises:
receiving signals captured by the external microphone;
determining the coherence between the signals detected by the implantable microphone and the signals detected by the external microphone; and
applying an equalization filter to the signals detected by the implantable microphone,
wherein coefficients of the equalization filter are updated based on the coherence between the signals detected by the implantable microphone and the signals detected by the external microphone.

5. The method of claim 4, further comprising:
adjusting a rate at which the coefficients of the equalization filter are updated based on the coherence between the signals detected by the implantable microphone and the signals detected by the external microphone.

6. (canceled)

7. The method of claim 4, wherein determining the coherence between the signals detected by the external microphone and the signals detected by the implantable microphone comprises:
determining a magnitude squared coherence between the signals detected by the external microphone and the signals detected by the implantable microphone.

8. The method of claim 1, wherein filtering vibration signals from the signals detected by the implantable microphone comprises:
determining the coherence between the signals detected by the implantable microphone and the signals detected by the implantable vibration sensor; and
applying a first body noise cancellation filter to the signals detected by the implantable microphone,
wherein coefficients of the first body noise cancellation filter are updated based on the coherence between the implantable sensor signals.

9. The method of claim 8, further comprising:
adjusting a rate at which the coefficients of the first body noise cancellation filter are updated based on the coherence between the signals detected by the implantable microphone and the signals detected by the implantable vibration sensor.

10. The method of claim 8, wherein both a magnitude and phase of the coefficients of the first body noise cancellation filter are adapted based on the coherence between the signals detected by the implantable microphone and the signals detected by the implantable vibration sensor.

11. The method of claim 8, wherein determining a coherence between the signals detected by the implantable microphone and the signals detected by the implantable vibration sensor comprises:
determining a magnitude squared coherence between the signals detected by the implantable microphone and the signals detected by the implantable vibration sensor.

12. The method of claim 8, further comprising:
applying a second body noise cancellation filter to the signals detected by the implantable microphone,
wherein the second body noise cancellation filter is applied after the first body noise cancellation filter.

13. (canceled)

14. (canceled)

15. The method of claim 1, wherein filtering vibration signals from the signals detected by the implantable microphone generates processed sounds signals, and wherein the method further comprises:
generating, based on the processed sounds signals, stimulation signals for delivery to the recipient to evoke perception by the recipient of acoustic sounds in the signals detected by the implantable microphone.

16. The method of claim 15, wherein generating stimulation signals for delivery to the recipient to evoke perception by the recipient of the acoustic sounds in the signals detected by the implantable microphone comprises:
generating electrical stimulation signals for delivery to the recipient evoke perception by the recipient of the acoustic sounds in the signals detected by the implantable microphone.

17. The method of claim 15, wherein generating stimulation signals for delivery to the recipient to evoke perception by the recipient of the acoustic sounds in the signals detected by the implantable microphone comprises:
generating acoustic stimulation signals for delivery to the recipient evoke perception by the recipient of the acoustic sounds in the signals detected by the implantable microphone.

18. An apparatus, comprising:
a first implantable sensor configured to capture signals comprising acoustic sounds and vibration signals;
a second implantable sensor configured to capture signals comprising at least vibration signals;
an implantable sound processing module configured to filter the vibration signals from the implantable sound signals based on a coherence between the signals captured by the first implantable sensor and the signals captured by the second implantable sensor to generate output signals; and
an implantable stimulator unit configured to generate, based on the output signals, stimulation signals for delivery to a recipient of the apparatus to evoke perception by the recipient of the acoustic sounds.

19. The apparatus of claim 18, wherein the first implantable sensor is a microphone and the second implantable sensor is an accelerometer.

20. The apparatus of claim 18, wherein the implantable sound processing module is configured to equalize a response of the first implantable sensor to a response of an external microphone based on a magnitude squared coherence between the signals captured by the first implantable sensor and signals captured by the external microphone.

21. The apparatus of claim 20, wherein the implantable sound processing module is configured to:
determine the magnitude squared coherence between the signals captured by the external microphone and the signals captured by the first implantable sensor; and
apply an equalization filter to the signals captured by the first implantable sensor, wherein coefficients of the equalization filter are dynamically updated based on the magnitude squared coherence between the signals captured by the external microphone and the signals captured by the first implantable sensor.

22. The apparatus of claim 21, wherein a rate at which the coefficients of the equalization filter are updated is controlled based on the magnitude squared coherence between the signals captured by the external microphone and the signals captured by the first implantable sensor.

23. The apparatus of claim 18, wherein to filter the vibration signals from the implantable sound signals based on a coherence between the signals captured by the first implantable sensor and the signals captured by the second implantable sensor, the implantable sound processing module is configured to:
determining the coherence between the signals captured by the first implantable sensor and the signals captured by the second implantable sensor; and
apply a first body noise cancellation filter to the signals captured by the first implantable sensor,
wherein coefficients of the first body noise cancellation filter are updated based on the coherence between the signals captured by the first implantable sensor and the signals captured by the second implantable sensor.

24. The apparatus of claim 23, wherein the implantable sound processing module is configured to adjust a rate at which the coefficients of the first body noise cancellation filter are updated based on the coherence between the signals captured by the first implantable sensor and the signals captured by the second implantable sensor.

25. The apparatus of claim 23, wherein to determine a coherence between the signals captured by the first implantable sensor and the signals captured by the second implantable sensor, the implantable sound processing module is configured to:
determine a magnitude squared coherence between the signals captured by the first implantable sensor and the signals captured by the second implantable sensor.

26. The apparatus of claim 23, wherein the implantable sound processing module is configured to:
apply a second body noise cancellation filter to the signals captured by the first implantable sensor,
wherein the second body noise cancellation filter is applied after the first body noise cancellation filter.

27. The apparatus of claim 23, wherein the implantable sound processing module is configured to determine the coefficients of the first body noise cancellation filter using an adaptive feedback loop.

28. The apparatus of claim 23, wherein the implantable sound processing module is configured to directly calculate the coefficients of the first body noise cancellation filter.

29. (canceled)

30. (canceled)

31. One or more non-transitory computer readable storage media comprising instructions that, when executed by at least one processor, are operable to:
obtain signals detected by an implantable microphone configured to be implanted in a recipient;
obtain signals detected by an implantable vibration sensor configured to be implanted in the recipient; and
adaptively equalize a response of the implantable microphone to a response of an external microphone based on a coherence between the signals detected by the implantable microphone and the signals detected by the external microphone.

32. The non-transitory computer readable storage media of claim 31, wherein the instructions operable to adaptively equalize a response of the implantable microphone to a response of an external microphone comprise instructions operable to:
obtain signals captured by an external microphone;
determine the coherence between the signals detected by the external microphone and the signals detected by the implantable microphone; and
apply an equalization filter to the signals detected by the implantable microphone,
wherein coefficients of the equalization filter are updated based on the coherence between the signals detected by the external microphone and the signals detected by the implantable microphone.

33. The non-transitory computer readable storage media of claim 32, further comprising instructions operable to:
adjust a rate at which the coefficients of the equalization filter are updated based on the coherence between the signals detected by the external microphone and the signals detected by the implantable microphone.

34. The non-transitory computer readable storage media of claim 33, wherein only a magnitude of the coefficients of the equalization filter are updated based on the coherence between the signals detected by the external microphone and the signals detected by the implantable microphone.

35. The non-transitory computer readable storage media of claim 33, wherein the instructions operable to determine a coherence between the signals detected by the external microphone and the signals detected by the implantable microphone comprise instructions operable to:
determine a magnitude squared coherence between the signals detected by the external microphone signals and the signals detected by the implantable microphone.

36-43. (canceled)
